(19) United States
(12) Patent Application Publication
(10) Pub. No.: US 2009/0306745 A1
(43) Pub. Date: Dec. 10, 2009

(54) ELECTRODE ASSEMBLY FOR DELIVERING LONGITUDINAL AND RADIAL STIMULATION

(75) Inventors: John L. Parker, Roseville (AU); Dusan Milojevic, Wheelers Hill (AU)

Correspondence Address: CONNOLLY BOVE LODGE & HUTZ LLP, 1875 EYE STREET, N.W., SUITE 1100, WASHINGTON, DC, 20006, US

(73) Assignee: Cochlear Limited, Lane Cove (AU)

(21) Appl. No.: 12/349,462

(22) Filed: Jan. 6, 2009

Related U.S. Application Data
(60) Provisional application No. 61/041,185, filed on Mar. 31, 2008.

(51) Int. Cl. A61N 1/05

(52) U.S. Cl. 607/57; 607/137

(57) ABSTRACT

An elongate electrode assembly for a cochlear implant comprising a plurality of sections arranged longitudinally along a length of the elongate electrode assembly. The sections each comprise one or more electrodes. At least one of the plurality of sections comprise two or more radially-spaced electrodes. The electrodes of adjacent sections are electrically discontinuous and the elongate electrode assembly is capable of delivering electrical stimulation in any one or a combination of radial mode, longitudinal mode, and radial-longitudinal mode. The electrodes in adjacent sections may be longitudinally spaced apart to provide electrical discontinuity. Alternatively, the plurality of sections may be arranged in offset layers and the electrodes in adjacent sections may be transversely spaced apart.

### CROSS-REFERENCE TO RELATED APPLICATIONS

The present application claims the benefit of U.S. Provisional Patent Application 61/041,185; filed Mar. 31, 2008, which is hereby incorporated by reference herein.

### BACKGROUND

1. Field of the Invention

The present invention relates to a tissue-stimulating prosthesis and, more particularly, to an electrode assembly for a tissue-stimulating prosthesis.

2. Related Art

Delivery of electrical stimulation to appropriate locations within the body may be used for a variety of purposes. For example, functional electrical stimulation (FES) systems may be used to deliver electrical pulses to certain muscles of a recipient to cause a controlled movement of the limb of such a recipient.

One example of an FES system is a cochlear implant designed for the hearing impaired. Hearing loss, which may be due to many different causes, is generally of two types, conductive and sensorineural. In some cases, a person may have hearing loss of both types. Conductive hearing loss occurs when the normal mechanical pathways for sound to reach the hair cells in the cochlea are impeded, for example, by damage to the ossicles. Conductive hearing loss is often addressed with conventional hearing aids which amplify sound so that acoustic information can reach the cochlea.

In many people who are profoundly deaf, however, the reason for their deafness is sensorineural hearing loss. Sensorineural hearing loss occurs when there is damage to the inner ear or to the nerve pathways from the inner ear to the brain. Those suffering from sensorineural hearing loss are thus unable to derive suitable benefit from conventional hearing aids. As a result, hearing prostheses that deliver electrical stimulation to nerve cells of the recipient's auditory system have been developed to provide persons having sensorineural hearing loss with the ability to perceive sound. Such stimulating hearing prostheses include, for example, auditory brain stimulators and Cochlear™ prostheses (commonly referred to as Cochlear™ prosthetic devices, Cochlear™ implants, Cochlear™ devices, and the like; simply “cochlea implants” herein.) As used herein, the recipient's auditory system includes all sensory system components used to perceive a sound signal, such as hearing sensation receptors, neural pathways, including the auditory nerve and spiral ganglion, and parts of the brain used to sense sounds.

Most sensorineural hearing loss is due to the absence or destruction of the cochlea hair cells which transduce acoustic signals into nerve impulses. It is for this purpose that cochlear implants have been developed. Cochlear implants use direct electrical stimulation of auditory nerve cells to bypass absent or defective hair cells that normally transduce acoustic vibrations into neural activity. Such devices generally use an electrode assembly implanted into the scala tympani of the cochlea so that the electrodes may differentially activate auditory neurons that normally encode differential pitches of sound.

Auditory brain stimulators are used to treat a smaller number of recipients with bilateral degeneration of the auditory nerve. For such recipients, the auditory brain stimulator provides stimulation of the cochlear nucleus in the brainstem.

FES systems, such as, cochlear implants, typically use an electrode assembly to deliver the electrical stimulation. These electrode assemblies typically includes an electrode assembly comprising a plurality of electrodes longitudinal (i.e., lengthwise) spaced along the assembly. Such assemblies are thus limited to applying electrical stimulation in a longitudinal manner.

### SUMMARY

In one aspect of the invention an elongate electrode assembly for a cochlear implant is provided. The elongate electrode assembly comprises a plurality of sections arranged longitudinally along a length of the elongate electrode assembly, the sections each comprising one or more electrodes. At least one of the plurality of sections comprises two or more radially-spaced electrodes, wherein electrodes of adjacent sections are electrically discontinuous, and wherein the elongate electrode assembly is capable of delivering electrical stimulation in any one or a combination of radial mode, longitudinal mode, and radial-longitudinal mode.

In yet another aspect, a method for delivering a stimulating signal to auditory neural tissue in a cochlea by a stimulating medical device having a plurality of radially and longitudinally-spaced electrodes is disclosed. The method comprises delivering delivering a first stimulating signal in longitudinal mode, delivering a second stimulating signal in any one or a combination of radial mode and radial-longitudinal mode, wherein the first and second stimulating signals each have first and second stimulation profiles, respectively.

In another aspect, an elongate electrode array for delivering stimulating signals to auditory neural tissue in a cochlea is provided. The elongate electrode array comprises means for delivering a first stimulating signal in a longitudinal mode and means for delivering a second stimulating signal in any one or a combination of radial mode and radial-longitudinal mode.

### BRIEF DESCRIPTION OF THE DRAWINGS

Illustrative embodiments of the present invention are described herein with reference to the accompanying drawings, in which:

FIG. 1 is a perspective view of a cochlear implant in which embodiments of the present invention may be implemented;

FIG. 2 is a perspective view of an electrode assembly having sections of both longitudinally and radially-spaced electrodes, in accordance with an aspect of the present invention;

FIG. 3A is a perspective view of another electrode assembly comprising sections of radially-spaced electrodes, in accordance with an aspect of the present invention;

FIG. 3B is a partial cross-sectional view of the electrode assembly of FIG. 3A, in accordance with an aspect of the present invention;

FIG. 3C is a partial cross-section view of another embodiment of the electrode assembly of FIG. 3A in which the top surface of the electrodes is embossed, in accordance with an aspect of the present invention;

FIG. 4A is a perspective view of a telescoping electrode assembly in an expanded state, in accordance with an aspect of the present invention;

FIG. 4B is a perspective view of the telescoping electrode assembly of FIG. 4A in a collapsed state, in accordance with an aspect of the present invention;

FIG. 5 is a perspective view of a portion of another embodiment of a telescoping electrode assembly in an expanded state, in accordance with an aspect of the present invention;

FIGS. 6A-C are illustrates of exemplary different electrode shapes for the electrode assembly, in accordance with an aspect of the present invention;

FIG. 7 is a representation of an electrode assembly in which the electrodes are delivering radial and longitudinal stimulation in bipolar mode, in accordance with an aspect of the present invention;

FIG. 8A-C is a representation of an electrode assembly comprising longitudinally and radially-spaced electrodes delivering bipolar stimulating signals, in accordance with an aspect of the present invention;

FIGS. 9A-D are cross-sectional representations of radially-spaced electrodes in an electrode array delivering stimulating signals in radial mode, in accordance with an aspect of the present invention;

FIG. 10 illustrates exemplary timing patterns for delivering electrical stimulation in longitudinal and radial modes, in accordance with an aspect of the present invention; and

FIG. 11 illustrates exemplary timing patterns using different types of pulses for delivering electrical stimulation in longitudinal and radial modes, in accordance with an aspect of the present invention.

### DETAILED DESCRIPTION

Embodiments of the present invention are directed to an apparatus and method for an electrode assembly for use in a tissue-stimulating prosthesis. In an embodiment, the electrode assembly is configured to provide both radial and longitudinal stimulation. For example, in an embodiment, the electrode assembly may comprise a one or more longitudinally and radially-spaced electrodes. Each of these longitudinally and radially-spaced electrodes may be individually used by the prosthesis in applying stimulation. This may thus enable the electrode assembly to be used in applying more complex and flexible stimulation strategies and enhanced performance to the end-user.

Embodiments of the present invention are described herein primarily in connection with one type of hearing prosthesis, namely a Cochlear™ prostheses (commonly referred to as Cochlear™ prosthetic devices, Cochlear™ implants, Cochlear™ devices, and the like; simply “cochlea implants” herein.) Cochlear implants generally refer to hearing prostheses that deliver electrical stimulation to the cochlear of a recipient. As used herein, cochlear implants also include hearing prostheses that deliver electrical stimulation in combination with other types of stimulation, such as acoustic or mechanical stimulation. It would be appreciated that embodiments of the present invention may be implemented in any cochlear implant or other hearing prosthesis now known or later developed, including auditory brain stimulators, or implantable hearing prostheses that acoustically or mechanically stimulate components of the recipient's middle or inner ear.

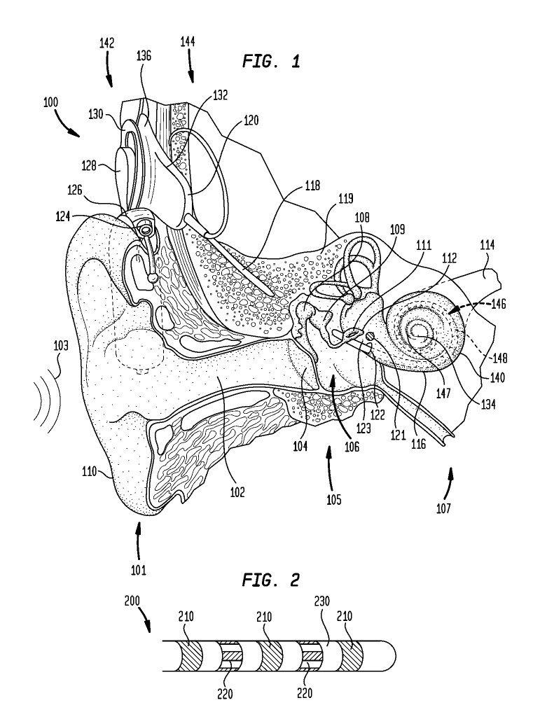

FIG. 1 is perspective view of a conventional cochlear implant, referred to as cochlear implant 100 implanted in a recipient having an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

Cochlear implant 100 comprises an external component 142 which is directly or indirectly attached to the body of the recipient, and an internal component 144 which is temporarily or permanently implanted in the recipient. External component 142 typically comprises one or more sound input elements, such as microphone 124 for detecting sound, a sound processing unit 126, a power source (not shown), and an external transmitter unit 128. External transmitter unit 128 comprises an external coil 130 and, preferably, a magnet (not shown) secured directly or indirectly to external coil 130. Sound processing unit 126 processes the output of microphone 124 that is positioned, in the depicted embodiment, by auricle 110 of the recipient. Sound processing unit 126 generates encoded signals, sometimes referred to herein as encoded data signals, which are provided to external transmitter unit 128 via a cable (not shown).

Internal component 144 comprises an internal receiver unit 132, a stimulator unit 120, and an elongate electrode assembly 118. Internal receiver unit 132 comprises an internal coil 136, and preferably, a magnet (also not shown) fixed relative to the internal coil. Internal receiver unit 132 and stimulator unit 120 are hermetically sealed within a biocompatible housing, sometimes collectively referred to as a stimulator/receiver unit. The internal coil receives power and stimulation data from external coil 130, as noted above. Elongate electrode assembly 118 has a proximal end connected to stimulator unit 120, and a distal end implanted in cochlea 140. Electrode assembly 118 extends from stimulator unit 120 to cochlea 140 through mastoid bone 119. Electrode assembly 118 is implanted into cochlea 104. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, sometimes referred to as electrode array 146 herein, disposed along a length thereof. As will be discussed in more detail below, in embodiments, electrodes may be radially and longitudinally spaced along this electrode array. Although electrode array 146 may be disposed on electrode assembly 118, in most practical applications, electrode array 146 is integrated into electrode assembly 118. As such, electrode array 146 is referred to herein as being disposed in electrode assembly 118. Stimulator unit 120 generates stimulation signals which are applied by electrodes 148 to cochlea 140, thereby stimulating auditory nerve 114.

In cochlear implant 100, external coil 130 transmits electrical signals (i.e., power and stimulation data) to internal coil 136 via a radio frequency (RF) link. Internal coil 136 is typically a wire antenna coil comprised of multiple turns of electrically insulated single-strand or multi-strand platinum or gold wire. The electrical insulation of internal coil 136 is provided by a flexible silicone molding (not shown). In use, implantable receiver unit 132 may be positioned in a recess of the temporal bone adjacent auricle 110 of the recipient.

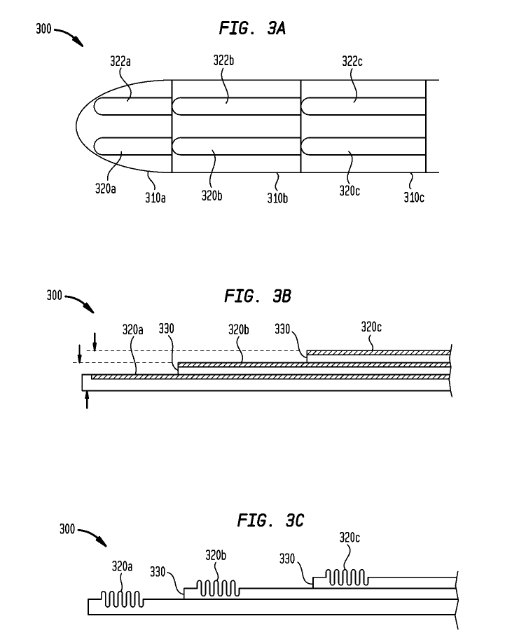

FIG. 2 depicts a portion of an electrode assembly 200. Electrode assembly portion 200 may be, for example, a portion of electrode assembly 118 of FIG. 1. As illustrated, electrode assembly portion 200 includes three single electrodes 210 and two sets of four radially-spaced electrodes 220. The electrodes 210, 220 are longitudinally spaced apart from one another and positioned on a flexible carrier to form the electrode assembly 118. As used herein, the term “electrode assembly” to refer to any type of assembly comprising a plurality of electrodes, such as, for example, any assembly comprising an array of electrodes.

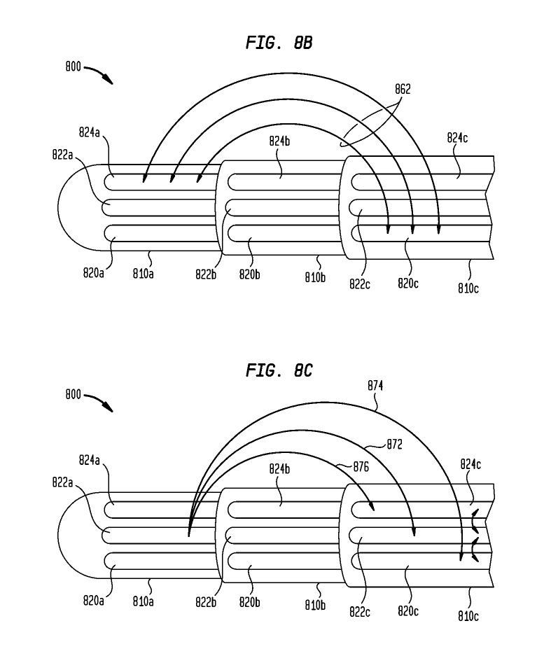

The longitudinal and radial space distribution between the stimulating surfaces of the electrodes 210, 220 enables the electrodes 210, 220 to deliver bipolar and/or tripolar electrical stimulation in at least three stimulation modes: longitudinal, radial and radial-longitudinal combined. Longitudinal stimulation may be delivered by two or more electrodes that are longitudinally separated along a length of the electrode array. In contrast, radial stimulation may be delivered by two or more electrodes at the same longitudinal position on the electrode array, but radially-spaced along the width of the electrode array. Thus, while longitudinal stimulation is capable of stimulating the spiral ganglion cells at varying depths of the cochlea, radial stimulation is capable of stimulating spiral ganglion cells that are radially spaced apart at a given depth along the modiolar wall. In addition to the longitudinal and radial stimulation modes, a combined radial-longitudinal mode may be delivered by two or more sets of radially-spaced electrodes that are longitudinally spaced apart, as shown in FIGS. 8A-C, which will be discussed in further detail below.

The electrode assembly 200 of FIG. 2 thus may be used for delivering both bipolar and tripolar electrical stimulation in any number and combination of modes. For example, the radially-spaced electrodes 220 may deliver bipolar and tripolar electrical stimulation in radial mode by using two and three of the radially-spaced electrodes, respectively. In addition, one or more of the remaining radially-spaced electrodes 220 may be coupled to a second single electrode 210 or radially-spaced electrode 220 that is positioned at a longitudinal distance away to deliver stimulation in longitudinal or radial-longitudinal modes, respectively.

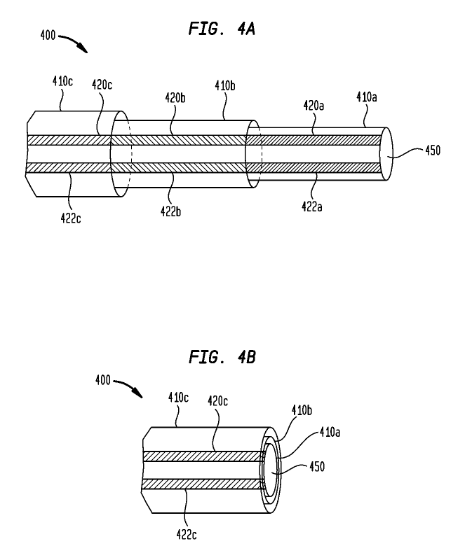

FIGS. 3A-B illustrate a portion of another embodiment of electrode assembly 118. FIG. 3A illustrates a top-down view of the portion 300 of the electrode assembly, and FIG. 3B illustrates a side-view of the portion 300. The illustrated portion of electrode assembly 300 comprises a plurality of sections 310a-c each comprising two radially-spaced electrodes 320a-c and 322a-c. Each radially-spaced electrode 320a-c and 322a-c may span the length of its respective section 310a-c. Further, in the embodiment of FIG. 3A-B, the electrode assembly 300 has a flat top surface upon/in which the electrodes 320a-c and 322a-c are housed. Accordingly, in this example, the radially-spaced electrodes are each located on the upper surface and separate from each other in the cross-wise direction (i.e., the direction essentially perpendicular to the lengthwise direction of the electrode assembly).

It should be noted that although FIGS. 3A-B illustrates an electrode assembly with a flat top surface in which the electrodes are embedded, in other embodiments the electrode assembly may be any other shape, such as for example, a cylindrical shape. Further, as used herein the term “radially-spaced” refers to spacing in which the electrodes are in different locations from each other in a direction other than parallel (e.g., perpendicular, or circumferential) to the lengthwise direction of the assembly. For example, in FIGS. 3A-B electrodes 320a and 322a are spaced apart from each other in a direction perpendicular to the lengthwise direction of the assembly and thus are considered radially-spaced. Similarly, electrodes 320b and 322c are spaced apart from each other in both a direction parallel to the lengthwise direction of the assembly and parallel to the lengthwise direction, and thus electrodes 320b and 322c are considered to be both radially-spaced and longitudinally spaced from each other. Further, referring back to FIG. 2, the electrodes 220 are spaced around the circumference of the assembly 200 (i.e., in a circumferential direction around lengthwise direction of the assembly) and thus are likewise considered radially-spaced. Additionally, as used herein, the term “section” refers to an area, part, or region of the assembly that is functionally or physically separate from the other sections of the assembly. For example, in the embodiment of FIGS. 3A-B, three separate sections 310a-c that are physically separate are illustrated. Similarly, in FIG. 2, each illustrated electrode 210 and group of radially-spaced electrodes 220 may be functionally separate from each other (i.e., use a different timing pattern or be independently controlled), and thus considered located in a different section of assembly 200.

As illustrated, adjacent sections 310a-c are layered, such that they are in slidable engagement with one another to provide a telescoping and collapsible electrode assembly 300. Electrodes of adjacent sections 310a-c are electrically discontinuous from each other in the present embodiment by use of an insulating layer 340a-c between each electrode. For example, each section may be manufactured from a material or materials (e.g., multiple layers of different materials) that provides electrode discontinuity between the electrodes, and then the electrodes may inserted into this material to form the sections 310a-c.

While FIGS. 3A-B depict each section has having a single continuous electrode pair, the section may also comprise any number of electrodes that are spaced apart and thus electrical discontinuous. As noted above, the electrical discontinuity within the section may be provided by an insulating layer.

In an embodiment, the stimulating surface of the electrodes may be processed to increase the surface area relative to its geometric size. This may be accomplished, for example, by embossing the exposed surface area of the electrodes. FIG. 3C illustrates a cross-section of the portion of the electrode assembly 300 of FIG. 3A where the surfaces of electrodes 320a-c and 322a-c (not illustrated in the view of FIG. 3C) have been embossed, as opposed to the flat electrodes 320a-c and 322a-c of FIG. 3B. It should be noted that this is but one example of an exemplary technique for increasing the electrode surface area and other methods may be used. High surface area electrodes with much smaller geometric surface areas than current designs may be used to either decrease the size of the electrode array or to increase the number of stimulating electrodes along the carrier.

In an embodiment, each electrode may be manufactured from a single piece of conductive material (e.g., platinum) such that stimulation pads (not illustrated) and conductive leads (not illustrated) for the electrode are in single continuous piece of platinum. Electrode assemblies in which the electrode simulating pads are integrated with the leads such that the electrodes are effectively an uncoated extension of the conductive lead are discussed in further detail in U.S. Pat. No. 7,240,416, the contents of which are incorporated herein by reference.

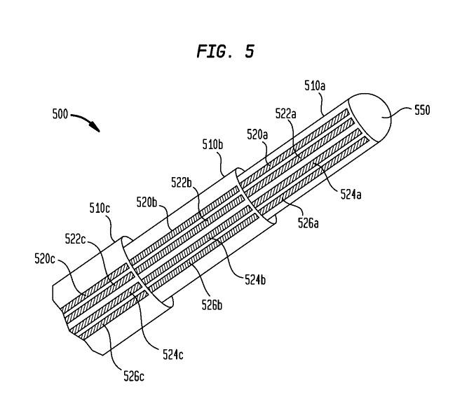

FIGS. 4A-B are perspective views of a portion 400 of yet another embodiment of the electrode assembly 118. As illustrated, in the embodiment of FIG. 4A-B, the electrode assembly is cylindrical in shape and comprised of layered sections 410a-c, which are in slidable engagement with respect to one another. The layered sections 410a-c each comprise two radially-spaced electrodes 420a-c and 422a-c which extend the length of each of their respective sections. As with the above-discussed embodiment of FIGS. 3A-B, electrical discontinuity between electrodes may be provided between the electrodes by, for example, using an insulating material or layer that separates the electrodes.

Electrode assembly 400 also includes a hollow lumen 450 through which a wire stylet may be inserted to aid in the positioning and implantation in the cochlea. The electrode assembly 400 may be advanced to the cochlea in the retracted state, as shown in FIG. 4B and the wire stylet may be used to deploy the electrode assembly 300 in an expanded state, as shown in FIG. 4A. Embodiments of telescoping electrode assemblies and methods of implantation in the cochlear are discussed in further detail in the co-pending U.S. patent application by John Parker, entitled “Telescoping Electrode Assembly,” filed concurrent with the present application, the contents of which are incorporated herein by reference.

While the portion of the electrode assembly 400 is depicted in FIGS. 4A-B as having a straight configuration, in embodiments, the electrode assembly may be biased to assume a pre-curved shape to conform to the shape of the cochlea. The deployment of pre-curved electrode assemblies may be accomplished in any number of ways. In one embodiment, a stylet may be used to hold a pre-curved electrode assembly in a generally straight configuration up until insertion. The stylet may be inserted into a lumen or channel located in the pre-curved electrode assembly with such lumen/channel allowing a passageway to accommodate the stylet. During or immediately following insertion, the stylet may be withdrawn allowing the assembly to return to its pre-curved configuration and assume a final position close to the inside wall of the cochlea.

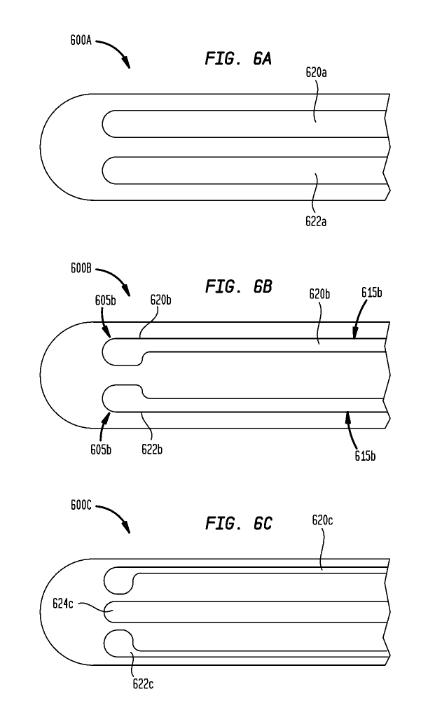

The electrode assembly disclosed herein may comprise any number of radially-spaced electrodes. FIG. 5, for example, illustrates a portion of an electrode assembly 500 including a plurality of sections 510a-c, each comprising four radially-spaced electrodes 520a-c, 522a-c. 524a-c, and 526a-c. Due to the size constraints of the electrode array, the electrodes 520a-c, 522a-c, 524a-c and 526a-c may each have a smaller geometric size relative to electrode assemblies having fewer electrodes. It may therefore be desirable to process the electrodes to increase their effective surface area, such as discussed above with reference to FIG. 3C (e.g., by embossing the surfaces of the electrodes).

An advantage of increasing the number of radially-spaced electrodes is that it may afford many different ways in which electrical stimulation may be delivered. Because the electrode array 500 has four radially-spaced electrodes in each section, the electrode array 500 may deliver bipolar stimulation in a variety of bipolar modes, such as between neighboring electrodes (e.g., 520a and 522a) or between nonadjacent electrodes (e.g., 520a and 524a or 520a and 526a). Bipolar stimulation between neighboring electrodes is referred to herein as BP+0, while bipolar stimulation between electrodes separated by one electrode is referred to as BP+1, bipolar stimulation between electrodes separated by two electrodes is referred to as BP+2, and so on. Wider stimulation modes (e.g., BP+1, BP+2, or greater) may be used to stimulate a greater number of cells.

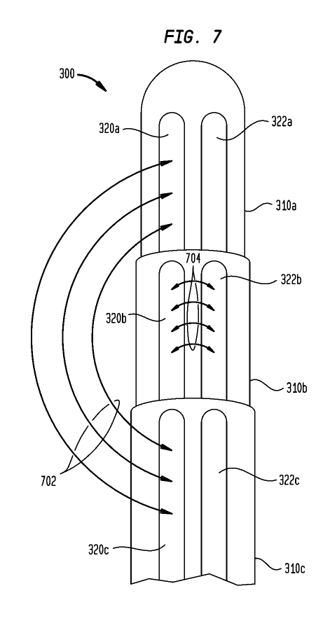

The delivery of stimulation signals to the cochlea may also be influenced by the shape of the stimulating electrodes. FIG. 6A-C provide simplified illustrations of portions of exemplary electrode assemblies in which the electrodes are configured in a variety of different shapes. Although the electrode assemblies of FIG. 6A-C are flat with the electrodes included in the top surface, it should be understood that the electrode assembly may be any shape (e.g., cylindrical or a custom design) and the electrodes may be included on any surface. Further, the illustrated shapes are exemplary only and the shape may be configured in any desired manner, such as, for example with a greater surface area provided in regions along the modiolus where it is desirable to have a greater intensity of electrical stimulation.

FIG. 6A depicts a tip of an electrode assembly 600A comprising a pair of radially-spaced electrodes 620A, 622A shaped as a pair of rods of constant widths. Thus, in this example, the intensity of the electrical stimulation delivered by the electrodes 620A, 620B is similarly uniform along the length of the electrode array. In FIG. 6B, the electrode assembly 600B comprises a pair of radially-spaced electrodes 620B, 622B in which a first end 605B is shaped to have a greater surface area than the second end 615B. Thus, the intensity of electrical stimulation delivered from the first end 605B of the radially-spaced electrodes 620B, 622B will be greater than that delivered by the second end 615B. While FIGS. 6A and 6B show the electrode array as having electrodes which are either identical (FIG. 6A) or mirror images of one another (FIG. 6B), the electrode assembly may include radially-spaced electrodes having completely different shapes and configurations. FIG. 6C is an example of such an electrode assembly 600C in which electrodes 620C, 622C are provided with a shape that is completely different from electrode 624C.

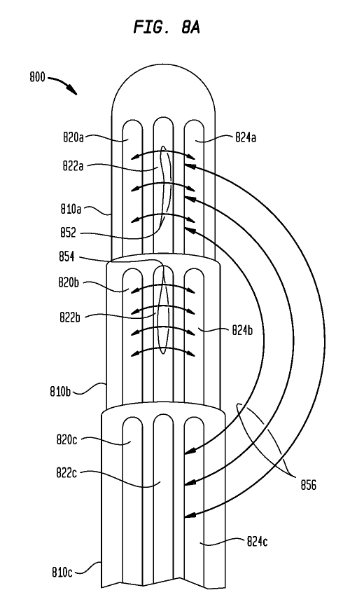

In application, the electrode assembly may use different stimulating modes for different purposes. FIG. 7 illustrates an exemplary stimulation modes that may be used for applying electrical stimulation using an electrode assembly. For exemplary purposes, FIG. 7 will be discussed below with reference to the electrode assembly of FIGS. 3A-B. As illustrated, electrode assembly 300 includes three sections 310a-c, with each of the three sections 310a-c having two radially-spaced electrodes 320a-c, 322a-c. As further shown, the two radially-spaced electrodes 320a-c, 322a-c are spaced apart in a longitudinal direction along the electrode assembly 300. FIG. 7 shows the electrode array 300 simultaneously delivering electrical stimulation in two stimulation modes: BP+1 in longitudinal mode 702 between electrodes 320a and 320c and BP+0 in radial mode 704 between electrodes 320b and 322b. As shown in FIG. 7, the radially-spaced electrodes are capable of delivering stimulation in both longitudinal and radial modes simultaneously.

FIGS. 8A-C depicts a part of an electrode assembly 800 having three sections 810a-c, with each of the three sections 810a-c having three radially-spaced electrodes 820a-c, 822a-c and 824a-c. FIGS. 8A-C will be used to provide an exemplary illustration of how different stimulation modes may be applied using the same electrode assembly 800. Electrode assembly 800 is similar to electrode assembly 200 of FIG. 2A-C and includes three sections 810a-c, but with each of the three sections 810a-c having three radially-spaced electrodes 820a-c, 822a-c, and 824a-c. As further shown, the three radially-spaced electrodes 820a-c, 822a-c, and 824a-c are spaced apart in a longitudinal direction along the electrode assembly 800.

FIG. 8A illustrates the electrode assembly 800 simultaneously delivering electrode stimulation in two simulation modes: BP+1 and BP+0. Particularly, as illustrated, BP+1 stimulation is delivered in radial mode 852 between electrodes 820a and 824a, and in radial mode 854 between electrodes 820b and 824b. BP+1 stimulation is delivered in longitudinal mode 856 between electrodes 822a and 822c. It is understood that the possible stimulation modes for the electrode arrays are not limited to those as shown in FIG. 8B. For example, because the electrode array in FIG. 8A-C has three radially-spaced electrodes, it is also capable of delivering tripolar stimulation in radial mode.

FIG. 8B depicts electrode assembly 800 delivering electrical stimulation in a combined radial-longitudinal modes. In FIG. 8B, the electrode assembly is depicted as delivering BP+1 radial-longitudinal stimulation 862 between electrodes 824a and 820c, where electrode 824a and 820c are located in different sections and in different radial locations from one another. As used herein, the term combined radial-longitudinal mode refers to a stimulation mode in which the stimulation is applied using electrodes having different longitudinal and radial locations.

FIG. 8C depicts electrode assembly 800 delivering stimulation in two modes simultaneously: (1) BP+1 radial-longitudinal mode stimulation between electrodes 622a and 620c and between electrodes 622a and 624c (illustrated by BP+1 radial-longitudinal stimulations 872, and 874, respectively) and (2) BP+1 longitudinal mode stimulation 876 between electrodes 622a and 622c. In an alternative embodiment, electrodes 620c, 622c and 624c may be interconnected (e.g., short connected) to constitute a single reference electrode such that the current from electrode 622a may spread to electrodes 620c, 622c and 624c. The type of stimulation (e.g., radial and/or longitudinal) and the timing and intensity of the stimulations applied by the electrode assembly may be determined by, for example, a sound processing unit, such as, for example, sound processing unit 126 of FIG. 1, and communicated to the internal stimulator unit which then applies the stimulation using the electrode assembly.

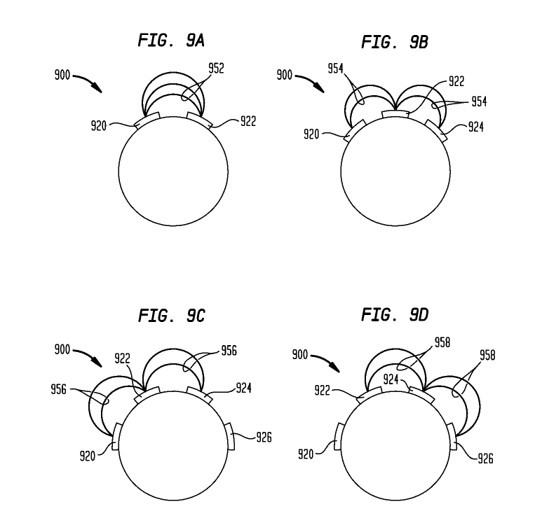

FIGS. 9A-D are cross sectional representations of electrode assemblies having two, three, and four radially-spaced electrodes delivering electrical stimulation in radial mode. FIG. 9A depicts an electrode assembly 900A including two electrodes 920 and 922 delivering bipolar stimulation (BP+0) 952 in radial mode. FIG. 9B depicts an electrode array 900B including three electrodes 920, 922 and 924 delivering tripolar stimulation 954 in radial mode. FIGS. 9C-D depict the electrode array 900C as having four electrodes 920, 922, 924, and 926 delivering tripolar stimulation in radial mode. Particularly, FIG. 9C illustrates tripolar stimulation 956 being delivered between electrode 922 and electrodes 920 and 924, and FIG. 9D illustrates tripolar stimulation 958 being delivered between electrode 924 and electrodes 922 and 926.

As noted above, in use, the electrode assembly is used to apply electrical stimulation to the user. Different strategies may be used by the system in applying this stimulation. This strategy may be included in software and/or hardware within the cochlear implant. For example, referring back to FIG. 1, the strategy may be included in software within the sound processing unit 126, which then determines the stimulation signals to be applied by each electrode as well as their timing. Data instructing the internal stimulation unit 120 to generate these stimulation signals to be applied by the electrode array may then be communicated to the stimulation unit, and the stimulation then applied to the user.

The following provides some example of some exemplary strategies that may be employed using a electrode assembly comprising longitudinally and radially spaced electrodes. In one simple example, stimulation may be applied using a single stimulation signal repeated over several electrodes of the electrode assembly. Or, for example, strategies may be used that provide improved stochastic and dispersed firing of the independent nerve fibers that more closely mimics the firings that occur naturally in healthy ears, such as the methods of delivering electrical stimulation to introduce dispersed and stochastic firing at more normal physiological rates disclosed in co-pending U.S. patent application Ser. No. 11/092,771, filed Mar. 30, 2005, the contents of which are incorporated herein by reference. Or, for example, alternative strategies for applying stimulation may be used. The below description discusses some exemplary stimulation strategies that may be used to applying stimulation in a system employing both radial and longitudinally spaced electrodes.

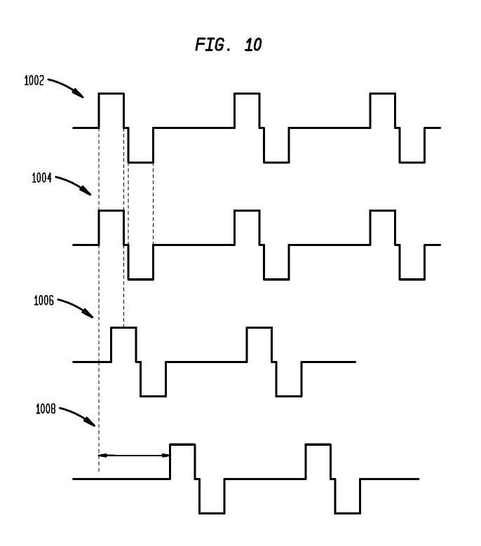

FIG. 10 illustrates exemplary timing patterns for delivering electrical stimulation in longitudinal and radial modes. The exemplary timing patterns of FIG. 10 use biphasic current pulses to generate the electrical stimulation provided by the electrodes. These exemplary biphasic current pulses are charge-balanced in this example to help prevent charge build-up within the cochlear tissues or the electrode bands as a result of stimulation. However, it should be understood that in other embodiments other pulse types (e.g., non-charge balanced) may be used depending on the situation. The perceived loudness of electrical stimulation generation using charge balanced biphasic pulses is generally related to the total charge delivered, with louder sounds produced by higher levels. The total charge, in turn, is determined by two pulse parameters: the pulse height and pulse width. These parameters may be manipulated to produce sufficiently loud stimuli in the shortest possible time.

Longitudinal stimulation using longitudinally spaced electrodes may be applied using a timing pattern such as timing pattern 1002, in which a positive signal (e.g., +1) is first applied for a brief period of time (e.g., 1 millisecond) followed almost immediately by a negative signal (e.g., −1) applied for a brief period of time (e.g., 1 millisecond), followed by a longer period of no stimulation (e.g., 5 millisecond), and then the pattern is repeated. It should be noted that the length of the signals and value of the signal applied are exemplary only and provided solely for explanatory purposes. Timing patterns 1004, 1006, and 1008 illustrate exemplary timing patterns that may be used for applying radial stimulation using the electrode array. Timing pattern 1004 illustrates a radial stimulation timing pattern that is identical to longitudinal stimulation timing pattern 1004, which accordingly results in simultaneous delivery of electrical stimulation in longitudinal and radial modes. Timing pattern 1006 illustrates a radial stimulation timing pattern that is identical to longitudinal stimulation timing pattern 1006 but is time-delayed (e.g., 0.1 millisecond) from the longitudinal stimulation timing pattern. Timing pattern 1008 illustrates a radial stimulation timing pattern that is identical to longitudinal stimulation timing pattern 1008 except that the radial stimulation is out-of-phase (e.g., it is delayed by half the distance between start and end of the repeated pattern, i.e., halfway between positive pulses, of the longitudinal timing pattern). In addition, the sequence of delivering electrical stimulation in longitudinal and radial modes may also be manipulated. It should be noted that the timing patterns of FIG. 10 are exemplary only and that other timing patterns may be used, such as for example, first applying radial stimulation which is then followed by longitudinal stimulation (e.g., radial stimulation is applied using timing pattern 1002 and longitudinal stimulation applied using timing patterns 1004, 1006, or 1008).

In addition to manipulating the timing of delivering electrical stimulations in longitudinal and radial modes, other stimulation parameters may be manipulated. For example, in embodiments the longitudinal stimulation may be applied using one type of pulse and radial stimulation applied using a different type of pulse.

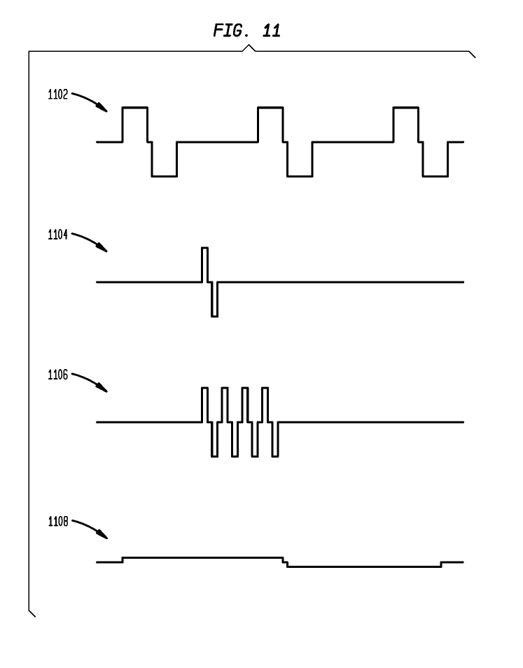

FIG. 11 provides exemplary longitudinal and radial stimulation timing patterns in which different types of pulses are used for applying longitudinal and radial stimulation using an electrode assembly comprising radial and longitudinally spaced electrodes.

Longitudinal timing pattern 1102 illustrates an exemplary timing pattern for applying longitudinal stimulation. For simplicity and explanatory purposes, timing pattern 1102 illustrated in FIG. 11 is identical to the above described longitudinal timing pattern 1002 of FIG. 10. Radial timing pattern 1104 illustrates a radial stimulation timing pattern that is identical to longitudinal stimulation timing pattern 1006 but is time-delayed (e.g., 3 milliseconds) from the longitudinal stimulation timing pattern and uses pulses of a different width (e.g., 1 millisecond). Radial timing pattern 1106 provides yet another exemplary timing pattern in which the timing pattern is delayed (e.g., 3 milliseconds) from the longitudinal timing pattern, uses pulses of a different width (e.g., 0.5 milliseconds) and employs 4 biphasic pulses in series followed by a period of no pulses before repeating the pattern. Radial timing pattern 1108 illustrates yet another exemplary radial stimulation timing pattern in which the stimulation is applied using a long positive pulse (e.g., 5 milliseconds) at one level (e.g., 0.2 volt) which then drops down to a second level (e.g., 0.1 volt) for a short duration of time (e.g., 1 millisecond). It should be noted that the above discussed timing patterns are exemplary only and provided to illustrate that a myriad of different timing patterns may be used for applying longitudinal and radial stimulation without departing from the invention.

The specific timing patterns used may be customizable to the end user and depend on the specific desired effect on the end user. For example, radial stimulation may be used to supply non-auditory stimulus, such as plasticity information stimulus, to the end user' and, longitudinal stimulation may used to deliver auditory information. The specific timing patterns used for radial stimulation and longitudinal stimulation may thus be selected based on the desired effect. Additionally, in yet another example, different timing patterns may be used based on the locations of the electrodes to be used. For example, it may be desirable to use longitudinal stimulation with electrodes located in one location of the cochlea, and, instead use radial stimulation with electrodes located in a different location of the cochlea.

It is to be understood that the detailed description and specific examples, while indicating embodiments of the present invention, are given by way of illustration and not limitation. Many changes and modifications within the scope of the present invention may be made without departing from the spirit thereof, and the invention includes all such modifications.

What is claimed is:

1. An elongate electrode assembly for a cochlear implant comprising:
a plurality of sections arranged longitudinally along a length of the elongate electrode assembly, the sections each comprising one or more electrodes;
at least one of the plurality of sections comprising two or more radially-spaced electrodes;
wherein electrodes of adjacent sections are electrically discontinuous; and
wherein the elongate electrode assembly is capable of delivering electrical stimulation in any one or a combination of radial mode, longitudinal mode, and radial-longitudinal mode.

2. The elongate electrode assembly of claim 1, further comprising an elongate carrier and wherein the sections are arranged along at least a part of the elongate carrier.

3. The elongate electrode assembly of claim 2, wherein the electrodes in adjacent sections are longitudinally spaced apart to provide the electrical discontinuity.

4. The elongate electrode assembly of claim 3, wherein the electrodes are longitudinally spaced apart by insulating material.

5. The elongate electrode assembly of claim 1, wherein the plurality of sections is arranged in offset layers.

6. The elongate electrode assembly of claim 5, a continuous stimulating surface is provided longitudinally along the length of the elongate electrode assembly.

7. The elongate electrode assembly of claim 6, wherein the electrodes of adjacent sections are transversely spaced apart.

8. The elongate electrode assembly of claim 1, wherein the plurality of sections each comprise two or more radially-spaced electrodes.

9. The elongate electrode carrier of claim 8, wherein adjacent electrodes within the section are configured to deliver bipolar stimulation in radial mode.

10. The elongate electrode carrier of claim 8, wherein the plurality of sections each comprise three or more radially-spaced electrodes.

11. The elongate electrode carrier of claim 10, wherein adjacent electrodes within the section are configured to deliver tripolar stimulation in radial mode.

12. The elongate electrode carrier of claim 8, wherein electrodes in adjacent sections are configured to deliver bipolar stimulation in any one or a combination of longitudinal mode and radial-longitudinal mode.

13. The elongate electrode assembly of claim 1, wherein the electrodes are treated to increase an effective surface area of the electrodes.

14. The elongate electrode assembly of claim 13, wherein the surface area of the electrodes is embossed.

15. A method for delivering a stimulating signal to auditory neural tissue in a cochlea by a stimulating medical device having a plurality of radially—and longitudinally—spaced electrodes, the method comprising:
delivering a first stimulating signal in longitudinal mode; and
delivering a second stimulating signal in any one or a combination of radial mode and radial-longitudinal mode;
wherein the first and second stimulating signals each have first and second stimulation profiles, respectively.

16. The method of claim 15, wherein the first and second stimulating signals are delivered simultaneously.

17. The method of claim 15, wherein the delivery of the first and second stimulating signals are separated by a time lapse.

18. The method of claim 15, wherein the first and second stimulating signals are delivered out of phase.

19. The method of claim 17, wherein the first stimulating signal is delivered after the second stimulating signal.

20. The method of claim 17, wherein the second stimulating signal is delivered after the first stimulating signal.

21. The method of claim 15, wherein the first stimulating signal comprises auditory information.

22. The method of claim 15, wherein the second stimulating signal comprises plasticity information.

23. The method of claim 15, wherein the first and second profiles each comprises parameters for pulse height, pulse width and frequency.

24. The method of claim 23, wherein at least one of the parameters for the first and second profiles are different.

25. An elongate electrode assembly for delivering stimulating signals to auditory neural tissue in a cochlear comprising:
means for delivering a first stimulating signal in longitudinal mode; and
means for delivering a second stimulating signal in any one or a combination of radial mode and radial-longitudinal mode.
